(19) United States
(12) Patent Application Publication
(10) Pub. No.: US 2019/0076006 A1
(43) Pub. Date: Mar. 14, 2019

(54) MEDICAL OBSERVATION DEVICE AND MEDICAL OBSERVATION SYSTEM

(71) Applicant: Sony Olympus Medical Solutions Inc., Tokyo (JP)

(72) Inventor: Takayuki TAKEDA, Tokyo (JP)

(73) Assignee: Sony Olympus Medical Solutions Inc., Tokyo (JP)

(21) Appl. No.: 16/114,935

(22) Filed: Aug. 28, 2018

(30) Foreign Application Priority Data
Sep. 7, 2017 (JP) 2017-171681

(51) Int. Cl. A61B 1/045; A61B 1/05; A61B 1/06; A61B 1/00

(52) U.S. Cl. CPC A61B 1/045; A61B 1/05; A61B 1/0676; A61B 1/012; A61B 1/00096; A61B 1/00009; A61B 1/00163

(57) ABSTRACT

There is provided a medical observation device including: an imaging control unit configured to control an imaging device with a plurality of light sources that emit light having different wavelengths. The imaging control unit controls light emission of the plurality of light sources so that intensity of light emitted by the light source having a shortest wavelength out of intensity of light at a reference color temperature is greater than intensity of light emitted at the reference color temperature. The reference color temperature is a reference color temperature used to display a medical captured image captured by the imaging device.

### CROSS REFERENCE TO RELATED APPLICATIONS

This application claims the benefit of Japanese Priority Patent Application JP 2017-171681 filed Sep. 7, 2017, the entire contents of which are incorporated herein by reference.

### BACKGROUND

The present disclosure relates to a medical observation device and a medical observation system.

In recent years, medical observation devices which enlarge observation targets such as lesions for observation, for example, in order to support microsurgery like neurosurgical operations and to perform endoscopic surgery are used in the medical field. As medical observation devices, for example, medical observation devices with optical microscopes and medical observation devices with imaging devices functioning as electronic imaging-type microscopes are exemplified. Such a medical observation device with an optical microscope will be referred to as an “optical medical observation device” below. In addition, such a medical observation device with an imaging device will be referred to as an “electronic imaging-type medical observation device” or may be referred to simply as a “medical observation device” below. In addition, an image obtained by capturing an observation target using an imaging device included in a medical observation device will be referred to as a “medical captured image” below.

Electronic imaging-type medical observation devices are designed to obtain image quality equal to or higher than that of optical medical observation devices accompanied by high image quality of imaging devices and high resolution of display devices on which captured images are displayed. In addition, it is not necessary for users who use such electronic imaging-type medical observation devices (e.g., medical staff including operators, assistants of operators, etc.) to look into eyepieces of optical microscopes as in cases in which they use optical medical observation devices, and thus the users can move positions of imaging devices more freely. Thus, using electronic imaging-type medical observation devices is advantageous in that microsurgery and the like can be supported more flexibly, and thus use of electronic imaging-type medical observation devices has been progressing in the medical field.

Under such circumstances, technologies of controlling a light source of an imaging device included in a medical observation device have been developed. As a technology for an endoscope which prevents a tone of an image from changing even if brightness of illumination light is changed, for example, the technology disclosed in JP 2012-217485A described below is exemplified.

### SUMMARY

An endoscope using the technology disclosed in JP 2012-217485A, for example, prevents a tone of an image from changing by increasing intensity of illumination light of a color in an amount smaller than that of another color of light among red light, green light, and blue light or decreasing intensity of illumination light of a color in an amount greater than that of another color of light.

However, depending on an observation target of a medical observation device, for example, an inside of the body of a patient captured by the medical observation device, a favorable signal-noise ratio (S/N) of luminance which contributes to high frequency components, rather than fidelity of color reproduction resulting from prevention of the change in a tone of an image, brings better visibility of a medical captured image. In addition, when visibility of a medical captured image is improved, medical staff including operators can observe operative sites more precisely with less stress.

The present disclosure proposes a novel and improved medical observation device and medical observation system that can obtain medical captured images with higher visibility.

According to an embodiment of the present disclosure, there is provided a medical observation device including: an imaging control unit configured to control an imaging device with a plurality of light sources that emit light having different wavelengths. The imaging control unit controls light emission of the plurality of light sources so that intensity of light emitted by the light source having a shortest wavelength out of intensity of light at a set reference color temperature is greater than intensity of light emitted at the reference color temperature. The reference color temperature is a reference color temperature used to display a medical captured image captured by the imaging device.

In addition, according to an embodiment of the present disclosure, there is provided a medical observation system including: a medical observation device including an imaging control unit configured to control an imaging device with a plurality of light sources that emit light having different wavelengths; and a display device configured to display a medical captured image captured by the imaging device on a display screen. The imaging control unit of the medical observation device controls light emission of the plurality of light sources so that intensity of light emitted by the light source having a shortest wavelength out of intensity of light at a reference color temperature is greater than intensity of light emitted at the reference color temperature. The reference color temperature is a reference color temperature used to display a medical captured image captured by the imaging device.

According to an embodiment of the present disclosure, medical captured images with higher visibility can be obtained.

Note that the effects described above are not necessarily limitative. With or in the place of the above effects, there may be achieved any one of the effects described in this specification or other effects that may be grasped from this specification.

### BRIEF DESCRIPTION OF THE DRAWINGS

FIG. 1 is an explanatory diagram illustrating a first example of a configuration of a medical observation system according to an embodiment of the present disclosure;

FIG. 2 is an explanatory diagram illustrating a second example of the configuration of the medical observation system according to an embodiment of the present disclosure

FIG. 3 shows explanatory diagrams for describing an example of a configuration of an imaging device included in the medical observation device illustrated in FIG. 2;

FIG. 4 illustrates an example of light of a plurality of light sources of the imaging device controlled by the medical observation device according to an embodiment of the present disclosure;

FIG. 5 is a graph showing an example of spectral characteristics of an image sensor with a color filter that transmits red light, a color filter that transmits green light, and a color filter that transmits blue light attached thereto;

FIG. 6 illustrates an example of light of a plurality of light sources of the imaging device controlled by the medical observation device according to an embodiment of the present disclosure;

FIG. 7 is a functional block diagram illustrating an example of a configuration of a medical observation device according to an embodiment of the present disclosure;

FIG. 8 is an explanatory diagram for describing a first example of a hardware configuration of the medical observation device that can perform a process according to the control method according to an embodiment of the present disclosure;

FIG. 9 shows explanatory diagrams illustrating an example of the image sensor; and

FIG. 10 is an explanatory diagram for describing a second example of a hardware configuration of the medical observation device that can perform a process according to the control method according to an embodiment of the present disclosure.

### DETAILED DESCRIPTION OF THE EMBODIMENT(S)

Hereinafter, (a) preferred embodiment(s) of the present disclosure will be described in detail with reference to the appended drawings. Note that, in this specification and the appended drawings, structural elements that have substantially the same function and structure are denoted with the same reference numerals, and repeated explanation of these structural elements is omitted.

In addition, description will be provided below in the following order.
1. Medical observation system according to present embodiment and control method according to present embodiment
2. Program according to present embodiment

### Medical Observation System According to Present Embodiment and Control Method According to Present Embodiment

An example of a medical observation system according to the present embodiment will be described, and then a control method according to the present embodiment that can be applied to the medical observation system according to the present embodiment will be described below.

### [1] Configuration of Medical Observation System

### [1-1] Medical Observation System According to First Example

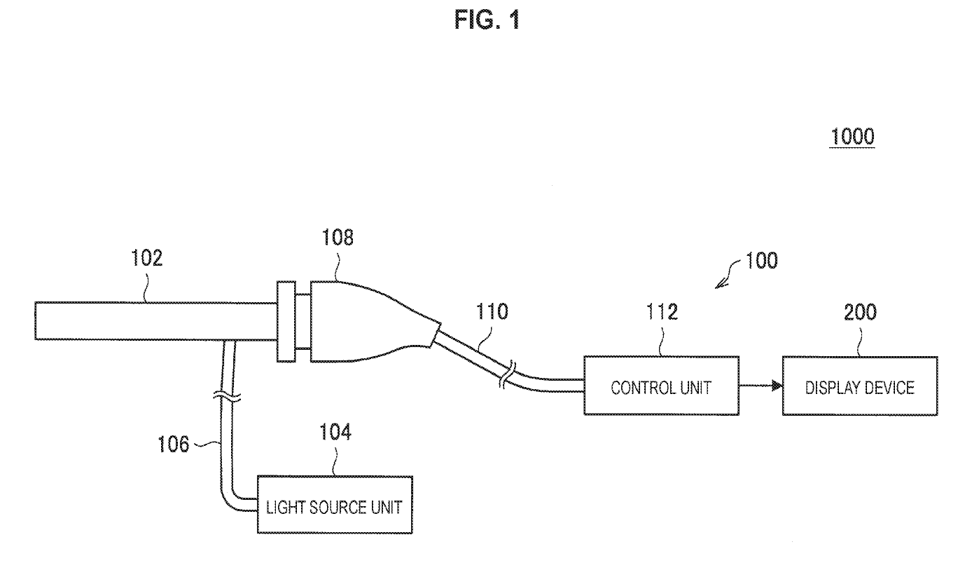

FIG. 1 is an explanatory diagram illustrating a first example of a configuration of a medical observation system 1000 according to the present embodiment, showing an example of a medical observation system having a medical observation device 100 that functions as an endoscope device that is an example of an electronic imaging-type medical observation device. The medical observation system 1000 illustrated in FIG. 1 has, for example, the medical observation device 100 and a display device 200.

Note that the medical observation system according to the first example is not limited to the example illustrated in FIG. 1.

The medical observation system according to the first example may further have a control device (not illustrated) that controls various operations of the medical observation device 100. An example in which the medical observation device 100 has the function of the control device (not illustrated) in the medical observation system 1000 illustrated in FIG. 1 when the medical observation device 100 has the control unit (which will be described below) that performs a process relating to the control method according to the present embodiment as will be described below is shown.

As the control device (not illustrated), for example, an arbitrary apparatus that can perform the process related to the control method according to the present embodiment such as a “medical controller,” or a “computer such as a server” is exemplified. In addition, the control device (not illustrated) may be, for example, an integrated circuit (IC) that can be incorporated into the above-described apparatus.

In addition, the medical observation system according to the first example may have a plurality of medical observation devices 100 and display devices 200. In a case in which a plurality of medical observation devices 100 are provided, each of the medical observation devices 100 performs the process related to the control method of the medical observation device 100 which will be described below. In addition, in the case in which the medical observation system according to the first example has a plurality of medical observation devices 100 and display devices 200, the medical observation devices 100 and the display devices 200 correspond to each other one to one, or the plurality of medical observation devices 100 may correspond to one display device 200. In the case in which the plurality of medical observation devices 100 correspond to one display device 200, for example, a switching operation is performed in the display device 200 to switch images captured by the medical observation devices 100 to be displayed on the display screen.

### [1-1-1] Display Device 200

The display device 200 is a display section of the medical observation system 1000, and corresponds to an external display device with respect to the medical observation device 100. The display device 200 displays various images, for example, medical captured images (moving images or a plurality of still images; the same applies below) captured by the medical observation device 100, images relating to a user interface, and the like. In addition, the display device 200 may be capable of performing 3D display. Display by the display device 200 is controlled by, for example, the medical observation device 100 or the control device (not illustrated).

The display device 200 of the medical observation system 1000 is installed in an arbitrary place at which the display device can be visually recognized by a person relating to surgery such as an operator within an operating room, for example, a wall surface, a ceiling, a floor of the operating room. As the display device 200, for example, a liquid crystal display, an organic electro-luminescence (EL) display, a cathode ray tube (CRT) display, or the like is exemplified.

Note that the display device 200 is not limited to the above-described example.

The display device 200 may be an arbitrary wearable device worn on the body of an operator or the like for use, for example, a head-mounted display, an eyewear-type device, or the like.

The display device 200 is driven by, for example, power supplied from an internal power supply included in the display device 200 such as a battery, power supplied from a connected external power supply, or the like.

### [1-1-2] Medical Observation Device 100

The medical observation device 100 constituting the medical observation system 1000 according to the first example is an endoscope device. In a case in which the medical observation device 100 illustrated in FIG. 1 is used during surgery, for example, an operator (an example of a user of the medical observation device 100) observes an operative site with reference to a medical captured image captured by the medical observation device 100 and displayed on the display screen of the display device 200, and performs various treatments such as a procedure on the operative site in accordance with a surgical technique.

The medical observation device 100 illustrated in FIG. 1 includes, for example, an insertion member 102, a light source unit 104, a light guide 106, a camera head 108, a cable 110, and a control unit 112. The medical observation device 100 is driven by, for example, power supplied from an internal power supply included in the medical observation device 100 such as a battery, power supplied from a connected external power supply, or the like.

The insertion member 102 has an elongated shape and has an optical system that collects incident light therein. A tip of the insertion member 102 is inserted into, for example, a body cavity of a patient. A rear end of the insertion member 102 is connected to a tip of the camera head 108 to be detachable therefrom. In addition, the insertion member 102 is connected to the light source unit 104 via the light guide 106 and thus receives supply of light from the light source unit 104.

The insertion member 102 may have, for example, a material having no flexibility or of a material having flexibility. The medical observation device 100 can be called a rigid endoscope or a flexible endoscope depending on a material forming the insertion member 102.

The light source unit 104 is connected to the insertion member 102 via the light guide 106. The light source unit 104 supplies light to the insertion member 102 via the light guide 106.

The light source unit 104 has a plurality of light sources that emit light having different wavelengths. As the plurality of light sources of the light source unit 104, for example, a light source that emits red light, a light source that emits green light, and a light source that emits blue light are exemplified. As the light source that emits red light, for example, one or two or more red light emitting diodes are exemplified. As the light source that emits green light, for example, one or two or more green light emitting diodes are exemplified. As the light source that emits blue light, for example, one or two or more blue light emitting diodes are exemplified. Note that the plurality of light sources of the light source unit 104 are not limited to the above-described examples, and may be arbitrary discrete light sources having discrete wavelengths. The light source unit 104 has, for example, the plurality of light sources in a single chip or in a plurality of chips.

The light source unit 104 is connected to the control unit 112 in a wired or wireless manner, and light emission performed by each of the plurality of light sources included in the light source unit 104 is individually controlled by the control unit 112.

Light supplied to the insertion member 102 is injected from the tip of the insertion member 102 and radiated to an observation target such as a tissue in a body cavity of a patient. In addition, light reflected from the observation target is collected by the optical system inside the insertion member 102.

The camera head 108 has a function of imaging an observation target. The camera head 108 is connected to the control unit 112 via the cable 110 that is a signal transmission member.

The camera head 108 includes an image sensor, captures the observation target by photoelectrically converting reflected light from the observation target collected by the insertion member 102, and outputs an image signal (a signal indicating a medical captured image) obtained from the imaging to the control unit 112 via the cable 110. As the image sensor of the camera head 108, for example, an image sensor using a plurality of image sensors such as complementary metal oxide semiconductors (CMOS), charge coupled devices (CCDs), and the like is exemplified.

In the medical observation device 100 functioning as an endoscope device, for example, the insertion member 102, the light source unit 104, and the camera head 108 play a role of an “imaging device that is inserted into the inside of the body of a patient and images the inside of the body.”

The control unit 112 plays a role of performing the process related to the control method according to the present embodiment and controls the imaging device. More specifically, the control unit 112 controls each of the light source unit 104 and the camera head 108. An example of a configuration of the control unit 112 that can control each of the light source unit 104 and the camera head 108 will be described below.

In addition, the control unit 112 includes a communication device (not illustrated), and transmits an image signal output from the camera head 108 to the display device 200 in arbitrary wireless communication or arbitrary wired communication. The control unit 112 may transmit an image signal and a display control signal to the display device 200.

As the communication device (not illustrated) included in the control unit 112, for example, an IEEE 802.15.1 port and a transmission/reception circuit (wireless communication), an IEEE 802.11 port and a transmission/reception circuit (wireless communication), a communication antenna and an RF circuit (wireless communication), an optical communication device (wired communication or wireless communication), a LAN terminal and a transmission/reception circuit (wired communication), or the like are exemplified. The communication device (not illustrated) may be capable of communicating with one or two or more external devices using a plurality of communication methods.

In addition, the control unit 112 may perform a predetermined process on the image signal output from the camera head 108 and transmit the image signal that has undergone the predetermined process to the display device 200. Examples of the predetermined process with respect to the image signal include adjustment of white balance, image enlargement or reduction in accordance with an electronic zoom function, inter-pixel correction, and the like.

Note that the control unit 112 may store the medical captured image on the basis of the image signal.

As the control unit 112, for example, a camera control unit (CCU) is exemplified.

The medical observation device 100 functioning as an endoscope device has, for example, a hardware configuration illustrated with reference to FIG. 1. In the medical observation device 100 functioning as an endoscope device, for example, the insertion member 102, the light source unit 104, and the camera head 108 play the role of an imaging device, and the control unit 112 controls imaging of the imaging device.

Note that the medical observation system 1000 according to the present embodiment is not limited to the configuration with the medical observation device 100 functioning as an endoscope device.

### [1-2] Medical Observation System According to Second Example

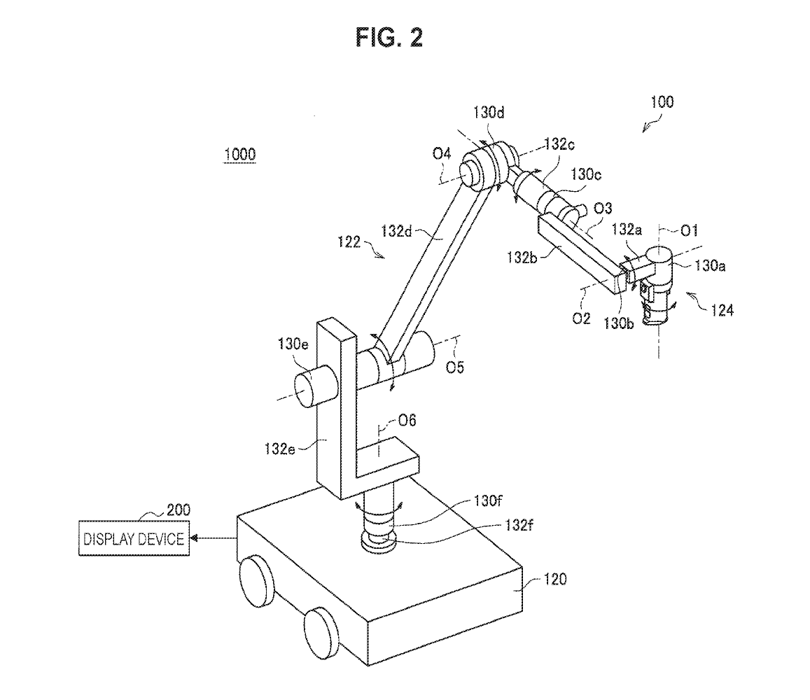

FIG. 2 is an explanatory diagram illustrating a second example of the configuration of the medical observation system 1000 according to the present embodiment, showing an example of the medical observation system having a medical observation device 100 functioning as an electronic imaging-type medical observation device according to another example. The medical observation system 1000 illustrated in FIG. 2 has, for example, the medical observation device 100 and a display device 200.

Note that the medical observation system according to the second example is not limited to the example illustrated in FIG. 2.

The medical observation system according to the second example may further have, for example, a control device (not illustrated) that controls various operations of the medical observation device 100, similarly to the medical observation system according to the first example.

In addition, the medical observation system according to the second example may have a plurality of medical observation devices 100 and a plurality of display devices 200, similarly to the medical observation system according to the first example.

### [1-2-1] Display Device 200

The display device 200 constituting the medical observation system according to the second example has a similar function and configuration to the display device 200 constituting the medical observation system according to the first example.

### [1-2-2] Medical Observation Device 100

The medical observation device 100 constituting the medical observation system 1000 according to the second example is an electronic imaging-type medical observation device according to another example. An example of a hardware configuration of the medical observation device 100 functioning as an electronic imaging-type medical observation device will be described with reference to FIG. 2.

The medical observation device 100 functioning as an electronic imaging-type medical observation device includes, for example, a base 120, an arm 122, and an imaging device 124.

In addition, although not illustrated in FIG. 2, the medical observation device 100 may also include, for example, one or two or more processors (not illustrated) constituted by an arithmetic circuit such as a micro-processing unit (MPU), a read only memory (ROM; not illustrated), a random access memory (RAM; not illustrated), and a recording medium (not illustrated), and a communication device (not illustrated). The medical observation device 100 is driven by, for example, power supplied from an internal power supply included in the medical observation device 100 such as a battery, power supplied from a connected external power supply, or the like.

The processors (not illustrated) function as a control unit which will be described below. The ROM (not illustrated) stores control data such as programs and arithmetic parameters to be used by the processors (not illustrated). The RAM (not illustrated) temporarily stores programs executed by the processors (not illustrated) and the like.

The recording medium (not illustrated) functions as a storage unit. The recording medium (not illustrated) stores, for example, various kinds of data such as data relating to the control method according to the present embodiment and various applications. Here, as the recording medium (not illustrated), for example, a magnetic recording medium such as a hard disk, a non-volatile memory such as a flash memory, or the like is exemplified. In addition, the recording medium (not illustrated) may be detachable from the medical observation device 100.

The communication device (not illustrated) is a communication section included in the medical observation device 100, and plays a role of performing wireless or wired communication with an external device such as the display device 200. Here, as the communication device (not illustrated), for example, an IEEE 802.15.1 port and a transmission/reception circuit, an IEEE 802.11 port and a transmission/reception circuit, a communication antenna and an RF circuit, an optical communication device, a LAN terminal and a transmission/reception circuit, or the like are exemplified. The communication device (not illustrated) may be capable of communicating with one or two or more external devices in a plurality of communication methods.

### [1-2-2-1] Base 120

The base 120 is the base of the medical observation device 100, and is connected to one end of the arm 122 to support the arm 122 and the imaging device 124.

In addition, the base 120 has, for example, casters, and the medical observation device 100 stands on the floor via the casters. By having the casters, the medical observation device 100 can be easily moved on the floor with the casters.

### [1-2-2-2] Arm 122

The arm 122 is constituted by a plurality of links connected to each other by joints.

In addition, the arm 122 supports the imaging device 124. The imaging device 124 supported by the arm 122 is three-dimensionally movable, and the arm 122 helps the imaging device 124 to maintain a position and a posture after movement.

More specifically, the arm 122 is constituted by, for example, a plurality of joints 130a, 130b, 130c, 130d, 130e, and 130f and a plurality of links 132a, 132b, 132c, 132d, 132e, and 132f that are connected by the joints 130a, 130b, 130c, 130d, 130e, and 130f to revolve. A rotatable range of each of the joints 130a, 130b, 130c, 130d, 130e, and 130f is arbitrarily set in the design stage, the manufacturing stage, or the like so that desired movement of the arm 122 is realized.

That is, in the medical observation device 100 illustrated in FIG. 2, six degrees of freedom with respect to movement of the imaging device 124 are realized by six rotation axes (a first axis O1, a second axis O2, a third axis O3, a fourth axis O4, a fifth axis O5, and a sixth axis O6) corresponding to the six joints 130a, 130b, 130c, 130d, 130e, and 130f constituting the arm 122. More specifically, in the medical observation device 100 illustrated in FIG. 2, movement of six degrees of freedom including three translational degrees of freedom and three rotational degrees of freedom is realized.

Each of the joints 130a, 130b, 130c, 130d, 130e, and 130f has an actuator (not illustrated), and each of the joints 130a, 130b, 130c, 130d, 130e, and 130f rotates at a corresponding rotational axis by driving of the actuator (not illustrated). Driving of the actuator (not illustrated) is controlled by, for example, a processor functioning as a control unit which will be described below or an external control device (not illustrated).

Since each of the joints 130a, 130b, 130c, 130d, 130e, and 130f rotates at a corresponding rotational axis by driving of the actuator (not illustrated), various kinds of operations of the arm 122, for example, stretching, shrinking (folding), and the like of the arm 122, are realized.

The joint 130a has a substantially cylindrical shape, and supports the imaging device 124 (an upper end portion of the imaging device 124 in FIG. 2) to be revolvable around a rotation axis (the first axis O1) parallel to a central axis of the imaging device 124 at a tip portion of the joints 130a (a lower end part thereof in FIG. 2). Here, the medical observation device 100 is configured such that the first axis O1 matches the optical axis of the imaging device 124. That is, by causing the imaging device 124 to revolve around the first axis O1 illustrated in FIG. 2, a medical captured image captured by the imaging device 124 becomes an image in which a line of sight is changed to rotate.

The link 132a is a substantially rod-shaped member, and fixedly supports the joint 130a. The link 132a extends, for example, in a direction orthogonal to the first axis O1 and is connected to the joint 130b.

The joint 130b has a substantially cylindrical shape and supports the link 132a to be revolvable around the rotation axis (the second axis O2) orthogonal to the first axis O1. In addition, the link 132b is fixedly connected to the joint 130b.

The link 132b is a substantially rod-shaped member and extends in a direction orthogonal to the second axis O2. In addition, the joint 130b and the joint 130c are respectively connected to the link 132b.

The joint 130c has a substantially cylindrical shape and supports the link 132b to be revolvable around the rotation axis (the third axis O3) orthogonal to the first axis O1 and the second axis O2. In addition, one end of the link 132c is fixedly connected to the joint 130c.

Here, by causing the tip side of the arm 122 (the side on which the imaging device 124 is provided) to revolve around the second axis O2 and the third axis O3, the imaging device 124 can be moved so that a position of the imaging device 124 is changed within a horizontal plane. That is, since rotation around the second axis O2 and the third axis O3 is controlled in the medical observation device 100, a line of sight of a medical captured image can be moved within a plane.

The link 132c has a member having one end in a substantially cylindrical shape and the other end in substantially a rod shape. The one end of the link 132c is fixedly connected to the joint 130c such that the central axis thereof and the central axis of the substantially cylindrical shape are the same. In addition, the other end of the link 132c is connected to the joint 130d.

The joint 130d has a substantially cylindrical shape and supports the link 132c to be revolvable around a rotational axis (the fourth axis O4) orthogonal to the third axis O3. The link 132d is fixedly connected to the joint 130d.

The link 132d is a substantially rod-shaped member and extends to be orthogonal to the fourth axis O4. One end of the link 132d is fixedly connected to the joints 130d to abut against a side face of the substantially cylindrical shape of the joint 130d. In addition, the other end of the link 132d (the end on the opposite side to the side on which the joint 130d is connected) is connected to the joint 130e.

The joint 130e has a substantially cylindrical shape and supports one end of the link 132d to be revolvable around the rotational axis (the fifth axis O5) parallel to the fourth axis O4. In addition, the joint 130e is connected to one end of the link 132e.

Here, the fourth axis O4 and the fifth axis O5 are rotational axis that can move the imaging device 124 in the vertical direction. By causing the tip side of the arm 122 (the side on which the imaging device 124 is provided) to revolve around the fourth axis O4 and the fifth axis O5, a position of the imaging device 124 in the vertical direction is changed. Thus, by causing the tip side of the arm 122 (the side on which the imaging device 124 is provided) to revolve around the fourth axis O4 and the fifth axis O5, a distance between the imaging device 124 and an observation target such as an operative site of a patient or the like can be changed.

The link 132e is a member constituted by a combination of a first member having a substantially L shape with one side extending in the vertical direction and the other side extending in the horizontal direction and a rod-shaped second member extending vertically downward from a portion of the first member extending in the horizontal direction. A portion of the first member of the link 132e extending in the vertical direction is fixedly connected to the joint 130e. In addition, the second member of the link 132e is connected to the joint 130f.

The joint 130f has a substantially cylindrical shape and supports the link 132e to be revolvable around a rotational axis (the sixth axis O6) parallel to the vertical direction. In addition, the joint 130f is fixedly connected to the link 132f.

The link 132f is a substantially rod-shaped member and extends in the vertical direction. One end of the link 132f is connected to the joint 130f. In addition, the other end of the link 132f (the end on the opposite side to the side on which the joints 130f is connected) is fixedly connected to the base 120.

Since the arm 122 has the above-described configuration, six degrees of freedom with respect to movement of the imaging device 124 are realized in the medical observation device 100.

Note that a configuration of the arm 122 is not limited to the above-described example.

For example, a brake that regulates rotation of each of the joints 130a, 130b, 130c, 130d, 130e, and 130f may be provided in each of the joints 130a, 130b, 130c, 130d, 130e, and 130f of the arm 122. As a brake according to the present embodiment, for example, an arbitrary type of brake such as a mechanically driven brake or an electrically driven electromagnetic brake is exemplified.

Driving of the brake is controlled by, for example, a processor that functions as a control unit which will be described below or an external control device (not illustrated). Since driving of the brake is controlled, an operation mode of the arm 122 is set in the medical observation device 100. As operation modes of the arm 122, for example, a fixed mode and a free mode are exemplified.

Here, the fixed mode according to the present embodiment is an operation mode in which, for example, a position and a posture of the imaging device 124 are fixed by a brake regulating rotation at each rotational axis provided in the arm 122. When the arm 122 is the fixed mode, an operation state of the medical observation device 100 is a fixed state in which a position and a posture of the imaging device 124 are fixed.

In addition, the free mode according to the present embodiment is an operation mode in which, when the brake is released, each rotational axis provided in the arm 122 is freely rotatable. In the free mode, for example, a position and a posture of the imaging device 124 can be adjusted through a direct operation by an operator. Here, a direct operation according to the present embodiment means, for example, an operation in which an operator grabs the imaging device 124 with his or her hand and moves the imaging device 124 in person.

### [1-2-2-3] Imaging Device 124

The imaging device 124 is supported by the arm 122 and captures an observation target, for example, an operative site of a patient, or the like. Imaging by the imaging device 124 is controlled by, for example, a processor that functions as a control unit, which will be described below, or an external control device (not illustrated).

The imaging device 124 has a configuration corresponding to, for example, an electronic imaging-type microscope.

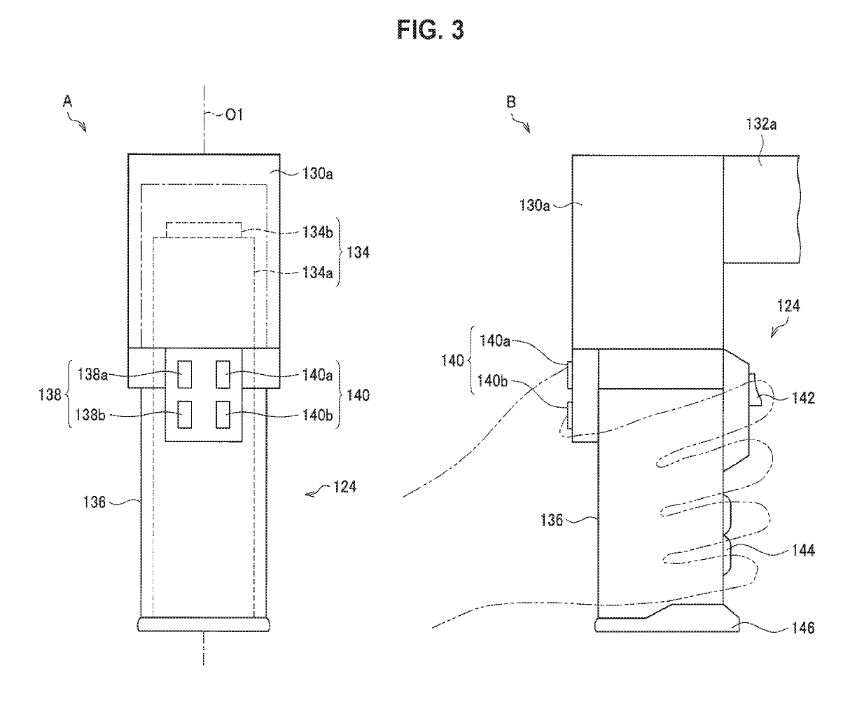

FIG. 3 shows explanatory diagrams for describing an example of a configuration of the imaging device 124 included in the medical observation device 100 illustrated in FIG. 2.

The imaging device 124 has, for example, an imaging member 134 and a tubular member 136 having a substantially cylindrical shape, and the imaging member 134 is provided in the tubular member 136.

Cover glass (not illustrated) for protecting the imaging member 134, for example, is provided on an opening surface of a lower end (an end on a lower side in FIG. 3) of the tubular member 136.

In addition, a plurality of light sources (not illustrated) that emit light having different wavelengths are provided, for example, inside of the tubular member 136, and during imaging, illumination light from the light source is radiated to a subject through the cover glass. Since reflected light (observation light) from the subject irradiated with the illumination light is incident on the imaging member 134 through the cover glass (not illustrated), an image signal (an image signal indicating a captured image) representing the subject is obtained by the imaging member 134.

As the plurality of light sources (not illustrated), a light source that emits red light, a light source that emits green light, and a light source that emits blue light, for example, are exemplified, similarly to the plurality of light sources of the light source unit 104 illustrated in FIG. 1. Note that the plurality of light sources (not illustrated) may be, for example, arbitrary discrete light sources having discrete wavelengths, similarly to the plurality of light sources of the light source unit 104 illustrated in FIG. 1. Light emission by each of the plurality of light sources is individually controlled by a processor (not illustrated) functioning as a control unit which will be described below.

The plurality of light sources are provided in a single chip or in a plurality of chips inside of the tubular member 136.

As the imaging member 134, a configuration used in any of various known electronic imaging-type microscope unit can be applied.

To give an example, the imaging member 134 is constituted by, for example, an optical system 134a and an image sensor 134b including an image sensor that captures image of an observation target using light that has passed through the optical system 134a. The optical system 134a includes optical elements, for example, one or two or more lenses such as an objective lens, a zoom lens, and a focus lens, a mirror, and the like. As the image sensor 134b, for example, an image sensor using a plurality of image sensors such as CMOS, CCDs, and the like is exemplified.

The imaging member 134 may have a pair of image sensors, that is, may function as a so-called stereo camera. The imaging member 134 may have one or two or more functions included in a general electronic imaging type microscope unit, such as a zoom function (one or both of an optical zoom function and an electronic zoom function), a focus function such as auto focus (AF), and the like.

In addition, the imaging member 134 may be capable of perform imaging at so-called high resolution of, for example, 4K, 8K, or the like. When the imaging member 134 can perform imaging at high resolution, it is possible to display an image on the display device 200 having a large display screen of, for example, 50 inches or greater while predetermined resolution (e.g., full HD image quality, etc.) is secured, and thus visibility of an operator viewing the display screen is improved. In addition, when the imaging member 134 can perform imaging at high resolution, even if a captured image is enlarged using the electronic zoom function and displayed on the display screen of the display device 200, predetermined resolution can be secured. Furthermore, in a case in which predetermined resolution is secured by using the electronic zoom function, performance of the optical zoom function of the imaging device 124 can be suppressed, and thus the optical system of the imaging device 124 can be made simpler and thus the imaging device 124 can be further miniaturized.

The imaging device 124 has, for example, various operation devices for controlling operations of the imaging device 124. In FIG. 3, for example, a zoom switch 138, a focus switch 140, and an operation mode change switch 142 are provided in the imaging device 124. Note that it is a matter of course that a position at which the zoom switch 138, the focus switch 140, and the operation mode change switch 142 are provided and shapes thereof are not limited to the example illustrated in FIG. 3.

The zoom switch 138 and the focus switch 140 are an example of an operation device for adjusting imaging conditions of the imaging device 124.

The zoom switch 138 is constituted by, for example, a zoom-in switch 124a for increasing zoom magnifications (enlargement magnifications) and a zoom-out switch 124b for decreasing zoom magnifications. A zoom magnification is adjusted by performing an operation on the zoom switch 138, and thereby zoom is adjusted. Increasing a zoom magnification may be referred to as “zoom in” and decreasing a zoom magnification may be referred to as “zoom out” below.

The focus switch 140 is constituted by, for example, a distant view focus switch 140a for lengthening a focal distance to an observation target (subject) and a near-view focus switch 140b for shortening a focal distance to an observation target. By adjusting a focal distance by performing an operation on the focus switch 140, focus is adjusted. Lengthening a focal distance to an observation target may be called “focus out,” and shortening a focal distance to an observation target may be called “focus in.”

The operation mode change switch 142 is an example of an operation device of the imaging device 124 for changing an operation mode of the arm 122. When an operation is performed on the operation mode change switch 142, the operation mode of the arm 122 is changed. As the operation mode of the arm 122, for example, there are the fixed mode and the free mode as described above.

As an example of an operation with respect to the operation mode change switch 142, an operation of pressing the operation mode change switch 142 is exemplified. For example, while an operator presses the operation mode change switch 142, the operation mode of the arm 122 shifts to the free mode, and when the operator does not press the operation mode change switch 142, the operation mode of the arm 122 shifts to the fixed mode.

In addition, in the imaging device 124, a non-slip member 144 and a projecting member 146, for example, are provided to improve operability, convenience, and the like during operations by an operator who performs an operation with respect to the various operation devices.

The non-slip member 144 is a member provided to prevent an operating body from slipping when, for example, an operator performs an operation on the tubular member 136 using an operating body such as his or her hand. The non-slip member 144 has, for example, a material having a high friction factor, and thus has a structure which makes it difficult for an operating body to slip due to unevenness.

The projecting member 146 is a member provided to prevent an operating body from blocking a visual field of the optical system 134a when an operator operates the tubular member 136 with the operating body such as his or her hand or to prevent the cover glass (not illustrated) from becoming dirty due to contact of the cover glass with an operating body when performing an operation with the operating body.

Note that it is a matter of course that a position at which each of the non-slip member 144 and the projecting member 146 is provided and a shape thereof are not limited to the example illustrated in FIG. 3. In addition, in the imaging device 124, one or both of the non-slip member 144 and the projecting member 146 may not be provided.

An image signal (image data) generated from imaging by the imaging device 124 is subject to, for example, image processing by a processor that functions as a control unit, which will be described below. As image processing according to the present embodiment, for example, adjustment of white balance, image enlargement or reduction in accordance with the electronic zoom function, inter-pixel correction, and the like are exemplified. Note that, in a case in which the medical observation system according to the second example has a control device (not illustrated) that controls various operations of the medical observation device 100, image processing according to the present embodiment may be performed by the control device (not illustrated).

The medical observation device 100 transmits, for example, a display control signal and an image signal that has undergone the above-described image processing to the display device 200.

When a display control signal and an image signal are transmitted to the display device 200, the display screen of the display device 200 displays a medical captured image obtained by capturing an observation target (e.g., a captured image in which an operative site is captured) enlarged or reduced to a desired magnification using one or both of the optical zoom function and the electronic zoom function.

The medical observation device 100 that functions as the electronic imaging-type medical observation device according to the other example has, for example, the hardware configuration illustrated with reference to FIGS. 2 and 3.

Note that a hardware configuration of the medical observation device that functions as the electronic imaging-type medical observation device according to the other example is not limited to the configuration illustrated with reference to FIGS. 2 and 3.

For example, the medical observation device according to the present embodiment may have the arm 122 that is directly installed on a ceiling, a wall surface, or the like of an operating room or the like, without having the base 120. For example, in a case in which the arm 122 is installed on a ceiling, the arm 122 of the medical observation device according to the present embodiment is hung from the ceiling.

In addition, although the example in which the arm 122 realizes six degrees of freedom with respect to driving of the imaging device 124 is illustrated in FIG. 2, a configuration of the arm 122 is not limited to the configuration in which driving of the imaging device 124 has six degrees of freedom. For example, the arm 122 may appropriately move the imaging device 124 in accordance with an application, and the number and disposition of the joints and links, directions of driving axes of the joints, and the like can be appropriately set so that the arm 122 has a desired degree of freedom. To give an example, the medical observation device according to the present embodiment may have a simpler configuration of controlling an X axis and a Y axis, like an ophthalmology microscope.

In addition, although the example in which various operation devices for controlling operations of the imaging device 124 are provided in the imaging device 124 is illustrated in FIGS. 2 and 3, some or all of the operation devices illustrated in FIGS. 2 and 3 may not be provided in the imaging device 124. To give an example, various operation devices for controlling operations of the imaging device 124 may be provided in a part other than the imaging device 124 constituting the medical observation device according to the present embodiment. In addition, to give another example, various operation device for controlling operations of the imaging device 124 may be external operation devices such as a foot switch and a remote controller.

As the medical observation device 100 constituting the medical observation system 1000 according to the present embodiment, for example, a medical observation device that functions as the endoscope device illustrated in FIG. 1, a medical observation device that functions as the electronic imaging-type medical observation device according to the other example illustrated in FIG. 2, or the like is exemplified.

### [2] Control Method According to Present Embodiment

### [2-1] Overview of Control Method According to Present Embodiment

As described above, depending on an observation target of a medical observation device, a favorable signal-noise ratio (S/N) of luminance which contributes to high frequency components, rather than fidelity of color reproduction resulting from prevention of a change in a tone of an image, brings better visibility of a medical captured image.

In addition, for example, in a case in which a light source that outputs illumination light having a continuous wavelength, such as xenon light or W-LED light, is used, setting illumination light of a light source having a continuous wavelength to pass through a filter to attenuate unnecessary wavelength components is considered as a method of changing balance of illumination wavelengths. However, the method of changing balance of illumination wavelengths is a method of relative wavelength selection, and it is not possible in the method of changing balance of illumination wavelengths to effectively use light output from a light source.

Therefore, in the medical observation system 1000, the medical observation device 100 controls the plurality of light sources of the imaging device and adjusts intensity of the spectrum of light emitted by the plurality of light sources (the process relating to the control method according to the present embodiment).

More specifically, the medical observation device 100 controls light emission of the plurality of light sources so that, out of intensity of light emitted by the plurality of light sources of the imaging device when a reference color temperature is realized, intensity of light emitted by the light source having the shortest wavelength is greater than intensity of light emitted when the reference color temperature is realized. Here, the reference color temperature is a reference color temperature used to display a medical captured image captured by the imaging device. The medical observation device 100 can increase the intensity of light emitted by the light source having the shortest wavelength more by making luminous flux of the light source having the shortest wavelength greater than luminous flux when the reference color temperature is realized.

As the reference color temperature according to the present embodiment, a color temperature corresponding to natural light such as sunlight is exemplified. The reference color temperature according to the present embodiment may be a fixed color temperature set in the design stage of the medical observation device 100 or the like beforehand or a variable color temperature changing due to an operation of a user of the medical observation device 100, or the like.

In the case in which the plurality of light sources are the light source that emits red light, the light source that emits green light, and the light source that emits blue light, the medical observation device 100 controls light emission of the plurality of light sources so that intensity of light emitted by the light source that emits blue light, which is the light source having the shortest wavelength, is greater than intensity of light emitted when the reference color temperature is realized.

In addition, the medical observation device 100 controls light emission of the plurality of light sources so that intensity of light emitted by the light sources other than the light source having the shortest wavelength among the plurality of light sources of the imaging device does not differ from the intensity of light emitted when the reference color temperature is realized.

In the case in which the plurality of light sources are the light source that emits red light, the light source that emits green light, and the light source that emits blue light, the medical observation device 100 controls light emission of the plurality of light sources so that intensity of light emitted by each of the light source that emits red light and the light source that emits green light is not changed.

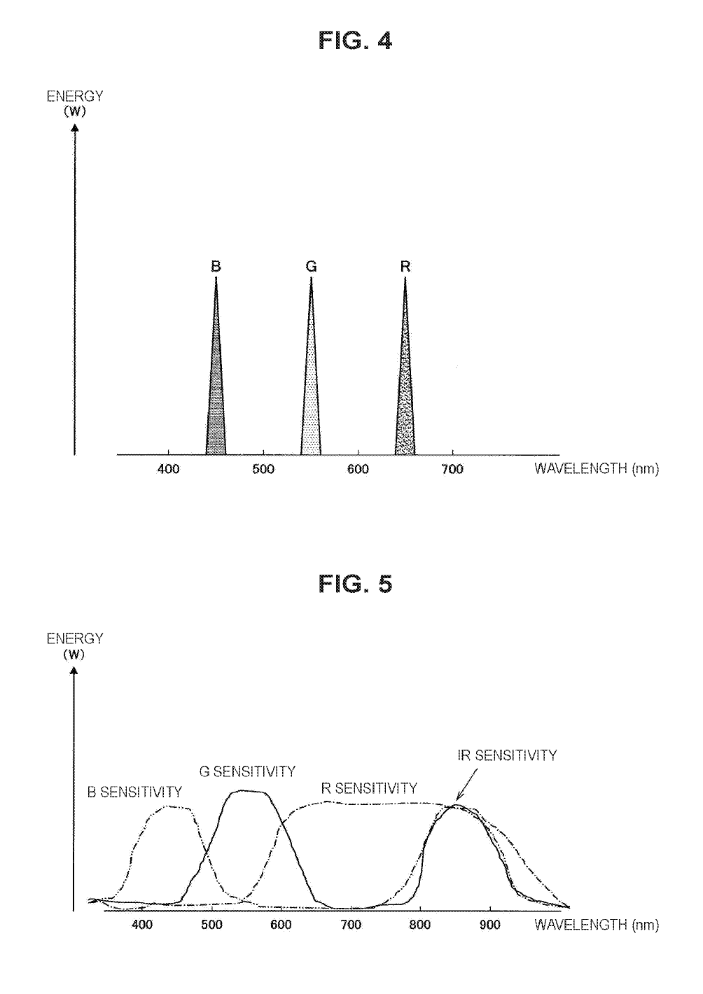

FIG. 4 illustrates an example of light of a plurality of light sources of the imaging device controlled by the medical observation device 100 according to the present embodiment. In FIG. 4, an example in which the plurality of light sources are a light source that emits red light, a light source that emits green light, and a light source that emits blue light is illustrated. In FIG. 4, red is denoted by “R,” green is denoted by “G,” and blue is denoted by “B” (the same applies to other drawings below).

As illustrated in FIG. 4, among wavelengths of light emitted by the plurality of light sources, the wavelength of blue light is the lowest and the wavelength of red light is the highest.

In the medical observation device 100, light emission of each of the plurality of light sources is individually controlled by the control unit 112 or the like, and intensity of light emitted by each of the plurality of light sources can be arbitrarily adjusted as described above. For example, in a case in which light from the light source emitting red light is strengthened, the illumination light is close to a fluorescence lamp having a high light rendering property, and in a case in which light from the light source emitting blue light is strengthened, the illumination light is close to sunlight.

A silicon-based image sensor used in observation of visible light discriminates wavelengths of incident light with color filters (e.g., a color filter that transmits red light, a color filter that transmits green light, and a color filter that transmits blue light) attached to a white and black image sensor. Although spectral sensitivity of the image sensor is designed to match the sensitivity of the eyes of a person viewing a captured image, luminance components that affect resolution are proportional to the number of pixels of the image sensor.

FIG. 5 is a graph showing an example of spectral characteristics of an image sensor with a color filter that transmits red light, a color filter that transmits green light, and a color filter that transmits blue light attached thereto. In FIG. 5, sensitivity of the image sensor to infrared light is denoted by “IR (Infrared) sensitivity.”

As illustrated in FIG. 5, for example, the image sensor used in observation of visible light has the lowest sensitivity to the wavelength of the blue region among the wavelength of the red region, the wavelength of the green region, and the wavelength of the blue region. The reason for this is that light having a short wavelength disappears in the course of photoelectric conversion by photodiodes constituting the image sensor. In such a silicon-based image sensor, for example, light having a higher wavelength reaches a deep part of the photodiodes and turns into electric charge.

Here, light-darkness of an object in a captured image contributes to a human's ability to sense brightness in the order of contribution levels of “green>red>blue.” In addition, although blue has a low level of contribution to a representative composition ratio of color components for luminance while green occupies the center, the composition ratio is proportional to the number of color filters attached to black and white pixels. In addition, with regard to an S/N of luminance that contributes to high frequency components, it is ideal for each of the colors to have the same S/N.

In the medical observation system 1000, the medical observation device 100 controls the plurality of light sources of the imaging device, controls light emission of the plurality of light sources so that, out of intensity of light emitted by the plurality of light sources when the reference color temperature is realized, the intensity of light emitted by the light source having the shortest wavelength is greater than the intensity of light emitted when the reference color temperature is realized, and the reference color temperature is a reference color temperature used to display a medical captured image captured by the imaging device as described above. Since light emission of the plurality of light sources is controlled as described above, the intensity of the spectrum of light emitted by the plurality of light sources is adjusted, and spectral sensitivity of the image sensor is corrected. As a result, although a color temperature of illumination light is changed, an S/N of luminance components is improved.

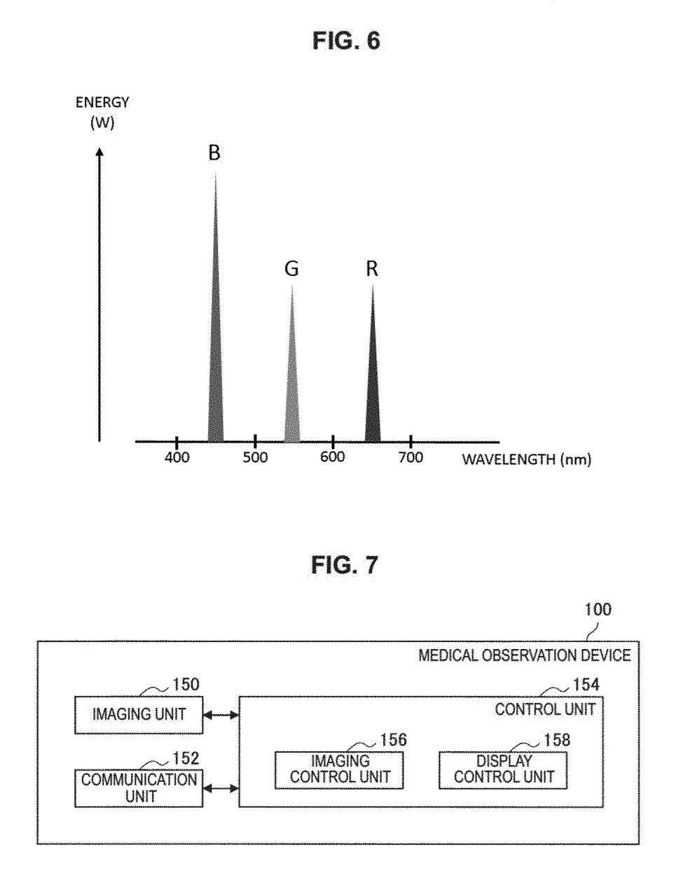

In a case in which the intensity of light when the reference color temperature is realized is the intensity of light illustrated in FIG. 4, for example, emission of blue light by the light source having the shortest wavelength among the plurality of light sources is controlled such that the intensity of the blue light emitted by the light source is greater than the intensity of light when the reference color temperature is realized as illustrated in FIG. 6.

In addition, the S/N of luminance that contributes to high frequency components brings better visibility of the medical captured image, rather than fidelity of color reproduction as described above.

Therefore, since the medical observation device 100 controls light emission of the plurality of light sources, the medical observation system 1000 that can obtain medical captured images with higher visibility is realized.

The medical observation system 1000 to which the control method according to the present embodiment is applied will be described along with description of functions of respective devices constituting the medical observation system 1000. In addition, a case in which the medical observation system 1000 according to the present embodiment is the medical observation system 1000 according to the first example illustrated in FIG. 1 will be mainly described below.

### [2-2] Medical Observation Device 100

FIG. 7 is a functional block diagram illustrating an example of a configuration of the medical observation device 100 according to the present embodiment. The medical observation device 100 includes, for example, an imaging unit 150, a communication unit 152, and a control unit 154.

### [2-2-1] Imaging Unit 150

The imaging unit 150 captures observation targets. In a case in which the medical observation device 100 has the configuration illustrated in FIG. 1, the imaging unit 150 is constituted by the insertion member 102, the light source unit 104, and the camera head 108 (members playing the role of an imaging device in the medical observation device 100 illustrated in FIG. 1). In addition, in a case in which the medical observation device 100 has the configuration illustrated in FIG. 2, the imaging unit 150 is constituted by the imaging device 124. Imaging performed by the imaging unit 150 is controlled by, for example, the control unit 154.

### [2-2-2] Communication Unit 152

The communication unit 152 is a communication section of the medical observation device 100, and plays the role of performing wireless or wired communication with an external device such as the display device 200. The communication unit 152 is constituted by, for example, the above-described communication device (not illustrated). Communication performed by the communication unit 152 is controlled by, for example, the control unit 154.

### [2-2-3] Control Unit 154

The control unit 154 plays a role of controlling the entire medical observation device 100. In addition, the control unit 154 plays the leading role of performing the process relating to the control method according to the present embodiment.

In the case in which the medical observation device 100 has the configuration illustrated in FIG. 1, the control unit 154 is constituted by, for example, the control unit 112. In addition, in the case in which the medical observation device 100 has the configuration illustrated in FIG. 2, the control unit 154 is constituted by, for example, the above-described processor (not illustrated). Note that the process relating to the control method by the control unit 154 may be distributed to be performed by a plurality of processing circuits (e.g., a plurality of processors and the like).

More specifically, the control unit 154 has, for example, an imaging control unit 156 and a display control unit 158.

### [2-2-3-1] Imaging Control Unit 156

The imaging control unit 156 controls light emission of the plurality of light sources so that, out of intensity of light emitted by the plurality of light sources of the imaging device when the reference color temperature is realized, the intensity of light emitted by the light source having the shortest wavelength is greater than the intensity of light emitted when the reference color temperature is realized as described above. Here, the reference color temperature is a reference color temperature used to display a medical captured image captured by the imaging device. In addition, the imaging control unit 156 controls light emission of the plurality of light sources so that intensity of light emitted by the light sources other than the light source having the shortest wavelength among the plurality of light sources of the imaging device does not differ from the intensity of light emitted when the reference color temperature is realized, as described above. That is, the imaging control unit 156 can set only the intensity of light emitted by the light source having the shortest wavelength among the plurality of light sources to be greater than the intensity of light emitted when the reference color temperature is realized.

In the case in which the plurality of light sources are a light source that emits red light, a light source that emits green light, and a light source that emits blue light, the imaging control unit 156 controls light emission of the light source that emits blue light so that intensity of light emitted by the light source that emits blue light is greater than the intensity of light emitted when the reference color temperature is realized. In addition, the imaging control unit 156 controls light emission of each of the light source that emits red light and the light source that emits green light so that intensity of light emitted by each of the light source that emits red light and the light source that emits green light is not changed.

Since light emission of the plurality of light sources is controlled as described above, for example, blue light (an example of light emitted by the light source having the shortest wavelength; the same applies below) is radiated more from the light source unit 104 than when the reference color temperature is realized.

Since sensitivity of the image sensor to the wavelength of the blue region is improved further by radiating blue light more than when the reference color temperature is realized, an S/N of luminance that contributes to high frequency components (Y_{H} which will be described below) is improved accordingly.

Meanwhile, by radiating blue light more than when the reference color temperature is realized, a color temperature of illumination light supplied to the insertion member 102 from the light source unit 104 is changed, and the color of an object actually observed under illumination light differs from the color of the object observed when the reference color temperature is realized. That is, since blue light is radiated more than when the reference color temperature is realized, fidelity of color reproduction decreases.

However, the S/N of luminance that contributes to high frequency components, rather than fidelity of color reproduction, brings better visibility of the medical captured image as described above.

Therefore, since the imaging control unit 156 controls light emission of the plurality of light sources as described above, a medical captured image with improved quality and high visibility can be obtained.

### [2-2-3-2] Display Control Unit 158

The display control unit 158 controls display of the display device 200 by, for example, transferring a display control signal and an image signal to the communication device (not illustrated) constituting the communication unit 152 and causing the display control signal and the image signal to be transmitted to the display device 200. Note that control over communication of the communication unit 152 may be performed by a communication control unit (not illustrated) constituting the control unit 154.

In addition, the display control unit 158 may control the color temperature of the display device 200 (an example of a display device that displays a medical captures image on a display screen) in accordance with the control of light emission of the plurality of light sources.

The display control unit 158 controls the color temperature of the display device 200 by transmitting state information indicating a state of the control of the light emission of the plurality of light sources to the display device.

As the state information according to the present embodiment, for example, data representing a color temperature of illumination light radiated as a result of control by the imaging control unit 156 over light emission of the plurality of light sources (e.g., data representing a color temperature of illumination light supplied from the light source unit 104 to the insertion member 102) is exemplified. The color temperature of illumination light is calculated using, for example, intensity of light emitted by each of the plurality of light sources. In addition, the color temperature of illumination light may be specified with reference to, for example, “a table (or a database) in which a combination of intensity of light emitted by each of the plurality of light sources is associated with a color temperature” stored in a recording medium (not illustrated) included in the medical observation device 100.

Note that state information according to the present embodiment is not limited to the data representing a color temperature of the radiated illumination light. For example, state information according to the present embodiment may be data representing intensity of light emitted by each of the plurality of light sources.

The display control unit 158 causes the state information to be transmitted to the display device 200 as metadata of an image signal (or a display control signal).

The display control unit 158 causes the state information to be transmitted to the display device 200 by superimposing the state information on an image signal (or a display control signal). To give an example, in a case in which the image signal (or the display control signal) is transmitted as a serial digital interface (SDI) signal, the display control unit 158 superimposes the state information as ancillary data in a blanking period of a synchronization signal (H_Sync or V_Sync). Note that a process relating to superimposition of the state information may be performed by a communication control unit (not illustrated) included in the control unit 154.

Note that data included in the metadata caused to be transmitted by the display control unit 158 is not limited to the above-described example. The metadata may include, for example, data representing an observation mode of the medical observation device 100. As the observation mode according to the present embodiment, for example, an observation mode in which imaging is performed with natural light, an observation mode in which imaging is performed using an image emphasis technology such as narrow band imaging (NBI), an observation mode in which imaging is performed with special light, and the like are exemplified. The special light according to the present embodiment is light in a specific wavelength band, for example, light in the near-infrared wavelength band, light in the fluorescence wavelength band for fluorescence observation using 5-ALA, or the like.

In the case in which the data representing an observation mode is included in the metadata, the display device 200, for example, analyzes the acquired metadata to process the image signal in accordance with the observation mode and thereby can cause a medical captured image to be displayed on the display screen.

The control unit 154 has a leading role of performing the process relating to the control method according to the present embodiment, for example, having the imaging control unit 156 and the display control unit 158.

Note that a configuration of the control unit 154 is not limited to the example illustrated in FIG. 7.

For example, the control unit 154 may not have the display control unit 158. Even in the case in which the display control unit 158 is not included, the medical observation device 100 can obtain a medical captured image with higher visibility using the imaging control unit 156 performing the process relating to the control method according to the present embodiment.

In addition, in the case in which the medical observation device 100 has the configuration illustrated in FIG. 2, for example, the control unit 154 may have an arm control unit (not illustrated) that controls driving of the arm 122. As an example of control of driving of the arm 122, for example, “application of a control signal for controlling driving to the actuator (not illustrated) corresponding to each of the joints 130a, 130b, 130c, 130d, 130e, and 130f” or the like is exemplified.

In addition, the control unit 154 can have an arbitrary configuration corresponding to a method of dividing the functions of the medical observation device 100, such as a configuration corresponding to a method of dividing the process relating to the control method according to the present embodiment, or the like.

By having the functions illustrated in, for example, FIG. 7, the medical observation device 100 performs the process relating to the control method according to the present embodiment.

Note that a configuration of the medical observation device according to the present embodiment is not limited to the configuration illustrated in FIG. 7.

For example, the medical observation device according to the present embodiment can have one or both of the imaging control unit 156 and the display control unit 158 illustrated in FIG. 7 separately from the control unit 154 (e.g., the imaging control unit 156 and the display control unit 158 can be realized as a separate processing circuit).

In addition, the medical observation device according to the present embodiment may not have the display control unit 158 illustrated in FIG. 7. Even in the case in which the display control unit 158 is not included, the medical observation device 100 can obtain a medical captured image with higher visibility by performing the process relating to the control method according to the present embodiment as described above.

In addition, a configuration of the medical observation device according to the present embodiment for realizing the process relating to the control method according to the present embodiment is not limited to the configuration illustrated in FIG. 7, for example, and the medical observation device according to the present embodiment can have a configuration corresponding to a method of dividing the process relating to the control method according to the present embodiment.

In addition, in a case in which communication is performed with an external device via an external communication device having a similar function and configuration to the communication unit 152, for example, the medical observation device according to the present embodiment may not include the communication unit 152.

In addition, in a case in which the medical observation system according to the present embodiment has a control device (not illustrated) and the medical observation device according to the present embodiment is controlled by the control device (not illustrated), the medical observation device according to the present embodiment may not include the control unit 154.

Here, when the control device (not illustrated) includes a control unit having a similar function and configuration to the control unit 154, for example, the control device performs the process relating to the control method according to the present embodiment and controls operations of each constituent element such as the imaging unit 150 or an arm unit (not illustrated) included in the medical observation device according to the present embodiment. When the control device (not illustrated) communicates with the medical observation device according to the present embodiment via the included communication device or a connected external communication device, the control device controls operations of each constituent element included in the medical observation device according to the present embodiment.

Furthermore, in a case in which the medical observation system according to the present embodiment has a control device (not illustrated) and the medical observation device according to the present embodiment is controlled by the control device (not illustrated), the medical observation device according to the present embodiment can also have a configuration without some of the functions of the control unit 154.

### [2-2-4] Example of Hardware Configuration of Medical Observation Device 100

Next, an example of a hardware configuration of the medical observation device 100 that can perform the process relating to the control method according to the present embodiment will be described.

### [2-2-4-1] First Example of Hardware Configuration of Medical Observation Device 100

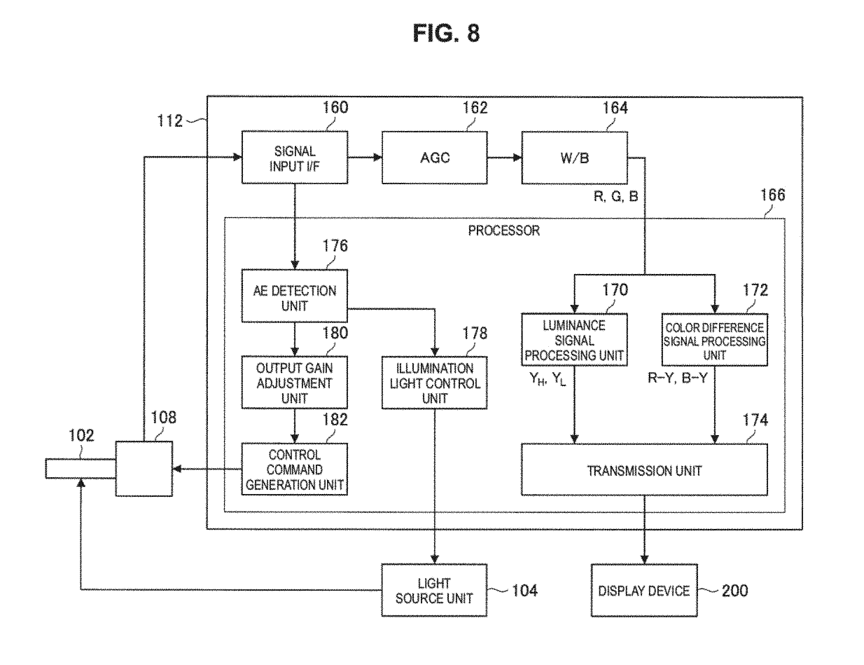

FIG. 8 is an explanatory diagram for describing a first example of a hardware configuration of the medical observation device 100 that can perform the process according to the control method according to the present embodiment, showing an example of a configuration of the control unit 112 illustrated in FIG. 1.

The control unit 112 has, for example, a signal input interface 160, an AGC circuit 162, a white balance circuit 164, and a processor 166 In FIG. 8, the signal input interface 160 is denoted by “signal input I/F,” the AGC circuit 162 is denoted by

“AGC (Automatic Gain Control),” and the white balance circuit 164 is denoted by “W/B.”

The signal input interface 160 is a communication interface to which signals are input, and image signals obtained from imaging by the camera head 108 are transferred to the signal input interface 160. As an image signal transferred from the camera head 108, for example, a signal indicating a raw image is exemplified.

An image signal input to the signal input interface 160 is subject to gain control in the AGC circuit 162 and to adjustment of white balance in the white balance circuit 164.

The white balance circuit 164 adjusts RGB balance when scattered reflected light from a white object is captured with the spectral sensitivity of the image sensor during balancing of a wavelength of radiated light, regardless of a light rendering index of illumination. That is, even in a case in which the processor 166, which will be described below, adjusts a color temperature of illumination light by controlling the light source unit 104 and thus increases an amount of light emitted by the light source having the shortest wavelength, white balance of a medical captured image is maintained through signal processing by the white balance circuit 164. Color balance of the medical captured image after white balance is adjusted is determined with, for example, the color temperature of the illumination light and the spectral sensitivity of the image sensor.

The image signal processed by the AGC circuit 162 and the white balance circuit 164 is processed by the processor 166.

The processor 166 has an arithmetic circuit such as an MPU and various processing circuits and plays a leading role of performing the process relating to the control method according to the present embodiment.

The processor 166 has, for example, a luminance signal processing unit 170, a color difference signal processing unit 172, a transmission unit 174, an AE detection unit 176, an illumination light control unit 178, an output gain adjustment unit 180, and a control command generation unit 182.

The luminance signal processing unit 170 performs arbitrary signal processing that contributes to improvement of image quality on, for example, a luminance signal on a basis of the image signal transferred from the AGC circuit 162. The luminance signal processing unit 170 separates the luminance signals from, for example, a high frequency component Y_{H} and a low frequency component Y_{L}, and performs high resolution processing on the high frequency component Y_{H} to improve resolution relating to image quality.

In a case in which, for example, a pixel array of the image sensor is Bayer array, the high frequency component Y_{H} is expressed by the following formula 1, and the low frequency component Y_{L} is expressed by the following formula 2. “R,” “G,” and “B” shown in the following formulas 1 and 2 each denote a “color signal corresponding to red light,” a “color signal corresponding to green light,” and a “color signal corresponding to blue light.”

Y_{H}=0.25×R+0.25×B+0.5×G (Formula 1)

Y_{L}=0.1×B+0.6×G+0.3×R (Formula 2)

As shown in the above formula 1, the “color signal corresponding to blue light” contributes to luminance of the high frequency component by the same amount as the “color signal corresponding to red light.” Thus, for luminance of the high frequency component, an S/N of the “color signal corresponding to blue light” is as important as that of the “color signal corresponding to red light.” In addition, when an observation target is a tissue inside an abdominal cavity, blue signal components may be a few, however, since color filters of the image sensor that transmit blue light account for ¼ of the total number of pixels, luminance for expressing resolution significantly contributes to the high frequency component.

On the other hand, the degree of contribution of the “color signal corresponding to blue light” to the low frequency component Y_{L} is small as shown in the above formula 2. In addition, since the degree of contribution of the “color signal corresponding to blue light” to the low frequency component Y_{L} is small, even a large amount of noise is inconspicuous.

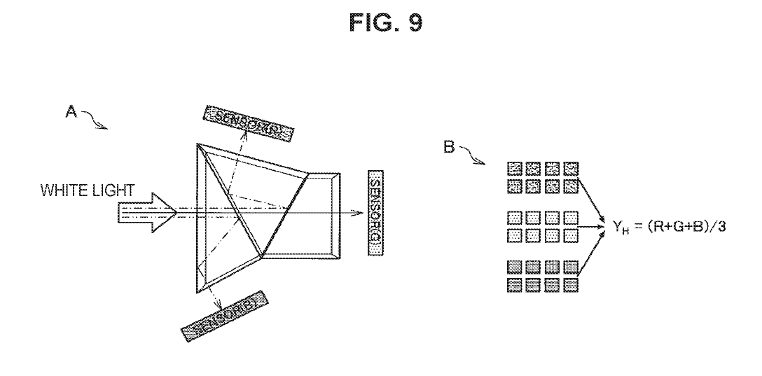

Note that the image sensor is not limited to an image sensor with Bayer array. FIG. 9 shows an explanatory diagrams illustrating an example of an image sensor, showing a three-plate-sensor-type image sensor. A of FIG. 9 conceptually shows a structure of the three-plate-sensor-type image sensor, and B of FIG. 9 shows an example of a calculation method of the high frequency component Y_{H} in the case of the three-plate-sensor-type image sensor.

In the case in which the image sensor is the three-plate-sensor-type image sensor, the high frequency component Y_{H} depends on special arrangement (allocation) of color filters each attached to the sensor (a color filter that transmits red light, a color filter that transmits green light, and a color filter that transmits blue light). In the example shown in A of FIG. 9, a “color signal corresponding to red light”, a “color signal corresponding to green light,” and a “color signal corresponding to blue light” are similarly treated. Thus, in the case in which the image sensor is the three-plate-sensor-type image sensor, the high frequency component Y_{H} can be expressed as shown in B of FIG. 9 by, for example, the following formula 3.

Y_{H}=(R+B+G)/3 (Formula 3)

Even in the case in which the high frequency component Y_{H} is expressed by the above formula 1, or the high frequency component Y_{H} is expressed by the above formula 3, a small amount of the “color signal corresponding to blue light” causes a decrease in an S/N of the high frequency component Y_{H}.

Meanwhile, the medical observation device 100 controls light emission of the light source that emits blue light so that the imaging control unit 156 sets intensity of light emitted by the light source that emits blue light to be greater than intensity of light emitted when the reference color temperature is realized, as described above. Thus, since the medical observation device 100 can increase the amount of the “color signal corresponding to blue light,” such a decrease in the S/N of the high frequency component Y_{H} is prevented.

The color difference signal processing unit 172 performs arbitrary signal processing that contributes to improvement of image quality on, for example, a color difference signal on the basis of the image signal transferred from the AGC circuit 162. The color difference signal is obtained by subtracting the luminance signal from the color signal.

The luminance signal is transferred from the luminance signal processing unit 170 to transmission unit 174, and the color difference signal is transferred from the color difference signal processing unit 172. Then, the transmission unit 174 transmits an image signal processed by each of the luminance signal processing unit 170 and the color difference signal processing unit 172 to the display device 200. The transmission unit 174, for example, transmits the signal-processed image signal obtained by performing signal processing on the luminance signal and the color difference signal in accordance with an output format. In addition, the transmission unit 174 may transmit a display control signal to the display device 200.

The AE detection unit 176 acquires an exposure detection value on the basis of the image signal input to the signal input interface 160. The exposure detection value can be calculated on the basis of, for example, a luminance value acquired from the image signal. The exposure detection range of the AE detection unit 176 may be a fixed range set in advance or a variable range based on an operation of a user on the medical observation device 100 or the like. The AE detection unit 176 transfers the acquired exposure detection value to each of the illumination light control unit 178 and the output gain adjustment unit 180.

The illumination light control unit 178 controls the light source unit 104 on the basis of the exposure detection value transferred from the AE detection unit 176 and thereby adjusts illumination light radiated from the light source unit 104.

In addition, the illumination light control unit 178 plays a leading role of performing the process relating to the control method by the above-described imaging control unit 156. That is, the illumination light control unit 178 controls the light source having the shortest wavelength so that, among the plurality of light sources of the light source unit 104, intensity of light emitted by the light source having the shortest wavelength is greater than intensity of light emitted when the reference color temperature is realized. In addition, the illumination light control unit 178 controls light emission of the other light sources so that intensity of light emitted by the light sources other than the light sources having the shortest wavelength does not differ from the intensity of light emitted when the reference color temperature is realized.

The output gain adjustment unit 180 changes an output gain of the image sensor on the basis of the exposure detection value transferred from the AE detection unit 176.

The control command generation unit 182 generates, for example, a command for controlling the camera head 108 on the basis of the change result of the output gain transferred from the output gain adjustment unit 180 and outputs the generated command to the camera head 108.

With the configuration of the control unit 112 illustrated in FIG. 8, for example, the process relating to the control method according to the present embodiment can be realized.

### [2-2-4-2] Second Example of Hardware Configuration of Medical Observation Device 100 and Example of Configuration of Display Device 200

Note that a configuration in which the process relating to the control method according to the present embodiment can be performed is not limited to the configuration illustrated in FIG. 8. The medical observation device 100 can control, for example, a color temperature of the display device 200 by causing state information in accordance with control of light emission of the plurality of light sources to be transmitted to the display device 200 as described above.

Therefore, as a second example of the hardware configuration of the medical observation device 100, an example of a hardware configuration of the medical observation device 100 that can cause state information to be transmitted to the display device 200 will be introduced. In addition, along with the second example of the hardware configuration of the medical observation device 100, an example of a configuration of the display device 200 in which a color temperature is controlled on the basis of state information will be introduced below.

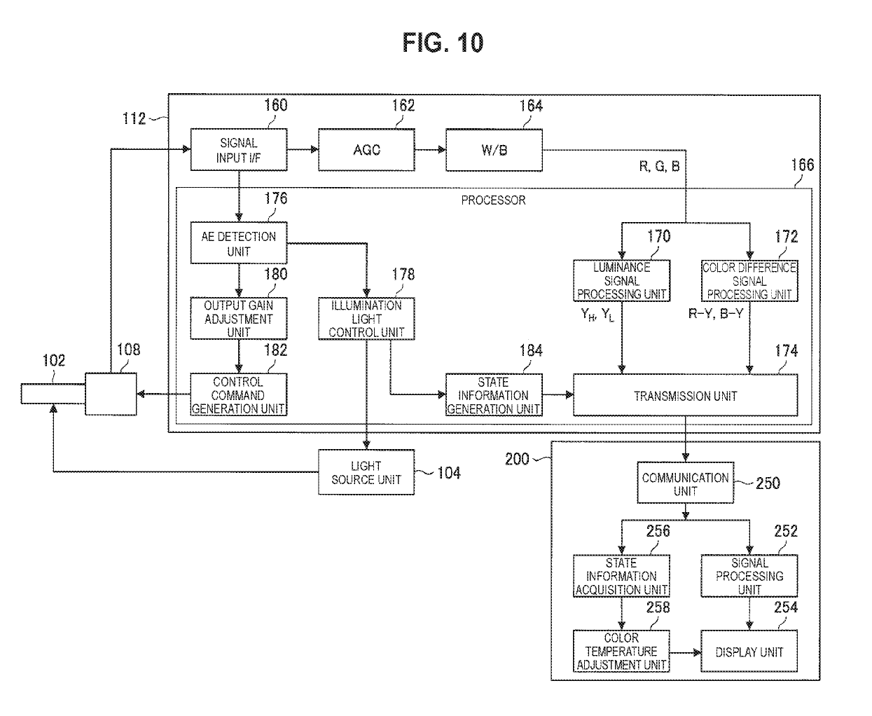

FIG. 10 is an explanatory diagram for describing the second example of the hardware configuration of the medical observation device 100 in which the process relating to the control method according to the present embodiment can be performed and the example of the configuration of the display device 200 according to the present embodiment. In FIG. 10, as the hardware configuration of the medical observation device 100 in which the process relating to the control method according to the present embodiment can be performed, a configuration of the control unit 112 illustrated in FIG. 1, similar to FIG. 8, is illustrated.

First, the second example of the hardware configuration of the medical observation device 100 will be described.

As illustrated in FIG. 10, the medical observation device 100 having the hardware configuration according to the second example basically has the similar hardware configuration of the medical observation device 100 according to the first example illustrated in FIG. 8. A difference of the medical observation device 100 illustrated in FIG. 10 from the medical observation device 100 illustrated in FIG. 8 is that the processor 166 of the medical observation device 100 illustrated in FIG. 10 further had a state information generation unit 184.

The state information generation unit 184 plays a leading role of performing the above-described process of controlling a color temperature of the display device 200 of the display control unit 158, and generates state information on the basis of intensity of light emitted by each of the plurality of light sources transferred from the illumination light control unit 178. The state information generation unit 184 calculates the color temperature of illumination light from, for example, the intensity of the light emitted by each of the plurality of light sources to acquire the color temperature of the illumination light, and then generates data representing the acquired color temperature of the illumination light as state information. In addition, the state information generation unit 184 may generate, for example, data representing intensity of light emitted by each of the plurality of light sources as state information.

Then, the state information generation unit 184 transmits the generated state information to the transmission unit 174. The state information generation unit 184 transmits the state information to the display device 200 by superimposing, for example, the state information on an image signal (or a display control signal) as described above.

Even in a case in which the control unit 112 has the hardware configuration illustrated in FIG. 10, for example, the process relating to the control method according to the present embodiment can be realized, as in the case in which the control unit 112 has, for example, the hardware configuration illustrated in FIG. 8. In addition, in the case in which the control unit 112 has the hardware configuration illustrated in FIG. 10, for example, since the state information in accordance with control of light emission by the plurality of light sources can be further transmitted to the display device 200, and thus the medical observation device 100 can control the color temperature of the display device 200.

Next, the example of the configuration of the display device 200 in which a color temperature is controlled on the basis of the state information will be described.

The display device 200 includes, for example, a communication unit 250, a signal processing unit 252, a display unit 254, a state information acquisition unit 256, a color temperature adjustment unit 258 as illustrated in FIG. 10. In the display device 200, for example, one or two or more processors included in the display device 200 function as the signal processing unit 252, the state information acquisition unit 256, and the color temperature adjustment unit 258.

The communication unit 250 is a communication section of the display device 200, and plays a role of performing wireless or wired communication with an external device of the medical observation device 100 or the like. The communication unit 250 includes, for example, an IEEE 802.15.1 port and a transmission/reception circuit, an IEEE 802.11 port and a transmission/reception circuit, a communication antenna and an RF circuit, an optical communication device, a LAN terminal and a transmission/reception circuit, or the like.

The signal processing unit 252 processes an image signal received by the communication unit 250. As the processing by the signal processing unit 252, for example, arbitrary signal processing such as high resolution processing is exemplified.

The display unit 254 includes, for example, a display panel, a light source, and various drivers, and displays an image corresponding to the image signal transferred from the signal processing unit 252 on a display screen. In addition, the drivers of the display unit 254 may operate on the basis of a display control signal received by the communication unit 250.

The state information acquisition unit 256 acquires the state information from the signal received by the communication unit 250. The state information acquisition unit 256 acquires the state information by, for example, separating ancillary data superimposed on the signal from the signal received by the communication unit 250.

The color temperature adjustment unit 258 adjusts a color temperature of the display device 200 on the basis of the acquired state information. In a case in which the acquired state information is “data representing a color temperature of illumination light,” for example, the color temperature adjustment unit 258 adjusts the color temperature of the display device 200 so that the color temperature of the illumination light represented by the data is corrected to the reference color temperature set in the display device 200. In addition, in a case in which the acquired state information is “data representing intensity of light emitted by each of the plurality of light sources,” for example, the color temperature adjustment unit 258 calculates a color temperature from the intensity of the light represented by the data. Then, the color temperature adjustment unit 258 adjusts the color temperature of the display device 200 so that the calculated color temperature is corrected to the reference color temperature set in the display device 200.

The color temperature adjustment unit 258 adjusts the color temperature of the display device 200 by, for example, controlling light emission of a light source (e.g., backlight, etc.) of the display unit 254.

Having the configuration illustrated in FIG. 10, for example, in the display device 200, the color temperature of the display device 200 is controlled on the basis of the acquired state information. By the display device 200 controlling the color temperature of the display device 200 on the basis of the state information, in the medical observation system 1000, a medical captured image with higher visibility is obtained and color reproducibility of the medical captured image displayed on the display screen of the display device 200 is improved.

Note that it is a matter of course that a configuration of the display device 200 in which the color temperature is controlled on the basis of the state information is not limited to the example illustrated in FIG. 10. For example, the reference color temperature may be set in the medical observation device 100 and the display device 200 may acquire the reference color temperature from the medical observation device 100, or the reference color temperature may be set in the display device 200 and the medical observation device 100 may acquire the reference color temperature from the display device 200.

### [3] Example of Effects Exhibited by Using Medical Observation System According to Present Embodiment

The following effects, for example, are exhibited by using the medical observation system according to the present embodiment. Note that it is a matter of course that an effect exhibited by using the medical observation system according to the present embodiment is not limited to the following examples.

In the image sensor, although sensitivity to the wavelength of the blue region is low among the wavelength of the red region, the wavelength of the green region, and the wavelength of the blue region, an S/N of luminance components can be relatively improved by increasing radiation intensity of blue light to increase an amount of a “color signal corresponding to blue light.”

By improving the S/N of luminance components, image quality resulting from signal processing can be improved, and thus images with higher quality can be created.

By improving the S/N of luminance components, it becomes hard to see noise components even if a gain of an aperture that uses visual characteristics of humans with respect to high frequency components of luminance or the like is increased.

Effective use of blue light (an example of light emitted by the light source having the shortest wavelength) as a mechanism for controlling illumination light to improve total image quality contributes to operators to alleviate stress by providing a medical captured image with less noise and further to perform surgery with good performance.

### Program According to the Present Embodiment

A medical captured image with higher visibility can be obtained by a processor and the like executing a program (e.g., a program that can execute the process relating to the control method according to the present embodiment) for causing a computer system to function as the medical observation device according to the present embodiment (or the control device according to the present embodiment) in the computer system. Here, as the computer system according to the present embodiment, a single computer or a plurality of computers are exemplified. A series of processes relating to the control method according to the present embodiment are performed by the computer system according to the present embodiment.

In addition, when the program for causing the computer system to function as the medical observation device according to the present embodiment (or the control device according to the present embodiment) is executed by the processor and the like in the computer system, an effect to be produced from display that is realized by the process relating to the above-described control method according to the present embodiment can be exhibited.

It should be understood by those skilled in the art that various modifications, combinations, sub-combinations and alterations may occur depending on design requirements and other factors insofar as they are within the scope of the appended claims or the equivalents thereof.

Although it has been described above that, for example, the program (computer program) for causing the computer system to function as the medical observation device according to the present embodiment is provided, the present embodiment can also provide a recording medium in which the program is stored therealong.

The above-described configuration is an example of the present embodiment, and of course belongs to the technical scope of the present disclosure.

Further, the effects described in this specification are merely illustrative or exemplified effects, and are not limitative. That is, with or in the place of the above effects, the technology according to the present disclosure may achieve other effects that are clear to those skilled in the art from the description of this specification.

Additionally, the present technology may also be configured as below.
(1) A medical observation device including:

an imaging control unit configured to control an imaging device with a plurality of light sources that emit light having different wavelengths,

in which the imaging control unit controls light emission of the plurality of light sources so that intensity of light emitted by the light source having a shortest wavelength out of intensity of light at a reference color temperature is greater than intensity of light emitted at the reference color temperature, and

the reference color temperature is a reference color temperature used to display a medical captured image captured by the imaging device.
(2) The medical observation device according to (1),

in which the plurality of light sources have a light source that emits red light, a light source that emits green light, and a light source that emits blue light, and

the imaging control unit controls light emission of the light source that emits blue light so that intensity of light emitted by the light source that emits blue light which is the light source having the shortest wavelength is greater than intensity of light emitted at the reference color temperature.
(3) The medical observation device according to (1) or (2), in which the imaging control unit controls light emission of the plurality of light sources so that intensity of light emitted by the light sources other than the light source having the shortest wavelength among the plurality of light sources does not differ from the intensity of light emitted at the reference color temperature.
(4) The medical observation device according to any one of (1) to (3), further including:

a display control unit configured to control display of the medical captured image captured by the imaging device,

in which the display control unit controls a color temperature of a display device that displays the medical captured image on a display screen in accordance with control of light emission by the plurality of light sources.
(5) The medical observation device according to (4), in which the display control unit controls the color temperature by causing state information indicating a state of the control of the light emission by the plurality of light sources to be transmitted to the display device.
(6) The medical observation device according to any one of (1) to (5), including:

the imaging device configured to be inserted into an inside of a body of a patient and capture the inside of the body.
(7) The medical observation device according to any one of (1) to (5), including:

an arm including a plurality of links connected to each other by a joint; and

the imaging device supported by the arm.
(8) A medical observation system including:

a medical observation device including an imaging control unit configured to control an imaging device with a plurality of light sources that emit light having different wavelengths; and

a display device configured to display a medical captured image captured by the imaging device on a display screen,

in which the imaging control unit of the medical observation device controls light emission of the plurality of light sources so that intensity of light emitted by the light source having a shortest wavelength out of intensity of light at a reference color temperature is greater than intensity of light emitted at the reference color temperature, and

the reference color temperature is a reference color temperature used to display a medical captured image captured by the imaging device.

What is claimed is:

1. A medical observation device comprising:
an imaging control unit configured to control an imaging device with a plurality of light sources that emit light having different wavelengths,
wherein the imaging control unit controls light emission of the plurality of light sources so that intensity of light emitted by the light source having a shortest wavelength out of intensity of light at a reference color temperature is greater than intensity of light emitted at the reference color temperature, and
the reference color temperature is a reference color temperature used to display a medical captured image captured by the imaging device.

2. The medical observation device according to claim 1,
wherein the plurality of light sources have a light source that emits red light, a light source that emits green light, and a light source that emits blue light, and
the imaging control unit controls light emission of the light source that emits blue light so that intensity of light emitted by the light source that emits blue light which is the light source having the shortest wavelength is greater than intensity of light emitted at the reference color temperature.

3. The medical observation device according to claim 1, wherein the imaging control unit controls light emission of the plurality of light sources so that intensity of light emitted by the light sources other than the light source having the shortest wavelength among the plurality of light sources does not differ from the intensity of light emitted at the reference color temperature.

4. The medical observation device according to claim 1, further comprising:
a display control unit configured to control display of the medical captured image captured by the imaging device,
wherein the display control unit controls a color temperature of a display device that displays the medical captured image on a display screen in accordance with control of light emission by the plurality of light sources.

5. The medical observation device according to claim 4, wherein the display control unit controls the color temperature by causing state information indicating a state of the control of the light emission by the plurality of light sources to be transmitted to the display device.

6. The medical observation device according to claim 1, comprising:
the imaging device configured to be inserted into an inside of a body of a patient and capture the inside of the body.

7. The medical observation device according to claim 1, comprising:
an arm including a plurality of links connected to each other by a joint; and
the imaging device supported by the arm.

8. A medical observation system comprising:
a medical observation device including an imaging control unit configured to control an imaging device with a plurality of light sources that emit light having different wavelengths; and
a display device configured to display a medical captured image captured by the imaging device on a display screen,
wherein the imaging control unit of the medical observation device controls light emission of the plurality of light sources so that intensity of light emitted by the light source having a shortest wavelength out of intensity of light at a reference color temperature is greater than intensity of light emitted at the reference color temperature, and
the reference color temperature is a reference color temperature used to display a medical captured image captured by the imaging device.
